(19) United States
(12) Patent Application Publication
(10) Pub. No.: US 2002/0146828 A1
(43) Pub. Date: Oct. 10, 2002

(54) Microparticles and methods for delivery of recombinant viral vaccines

(76) Inventors: John Hural, Bainbridge Island, WA (US); Mark E. Johnson, Bellevue, WA (US); A. Gregory Spies, Shoreline, WA (US)

Correspondence Address: GATES & COOPER LLP, Howard Hughes Center, Suite 1050, 6701 Center Drive West, Los Angeles, CA, 90045, US

(21) Appl. No.: 10/040,990

(22) Filed: Jan. 7, 2002

Related U.S. Application Data
(60) Provisional application No. 60/260,164, filed on Jan. 5, 2001.
(60) Provisional application No. 60/333,701, filed on Nov. 27, 2001.

(51) Int. Cl. C12N 15/86; C12N 7/00

(52) U.S. Cl. 435/456; 435/235.1; 435/320.1

(57) ABSTRACT

Disclosed is a viral vector conjugated to a microparticle, wherein the viral vector comprises a polynucleotide encoding a heterologous polypeptide. Conjugation of the viral vector to the microparticle results in a dramatic increase in the efficacy of the elicited immune response. Also disclosed is a method for delivering a polynucleotide to a cell comprising contacting the cell with a viral vector of the invention. In a preferred embodiment, the cell is an antigen-presenting cell, such as a dendritic cell. The invention further provides a vaccine comprising a viral vector of the invention. The invention thus provides a method for delivering a polynucleotide to a subject, a method of stimulating an immune response in a subject, a method of treating cancer in a subject, a method of inhibiting tumor growth in a subject, and a method of treating an infection in a subject.

This application claims the benefit of priority to U.S. provisional application Nos. 60/260,164, filed Jan. 5, 2001, and 60/333,701, filed Nov. 27, 2001, the entire contents of each of which are incorporate by reference herein.

Throughout this application various publications are referenced. The disclosures of these publications in their entireties are hereby incorporated by reference into this application in order to describe more fully the state of the art to which this invention pertains.

### TECHNICAL FIELD OF THE INVENTION

The invention relates to formulations, compositions and methods that can be used for the delivery of vaccines comprising virus particles, virus-like particles or virus replicon particles conjugated to microparticles. More particularly, the virus particles, virus-like particles or virus replicon particles include a polynucleotide that encodes an immunogenic polypeptide.

### BACKGROUND OF THE INVENTION

Recombinant virus-mediated gene transfer is an attractive method of gene delivery to some cell types, particularly in vitro. Advantages of viral systems include the large carrying capacity for recombinant transgenes, viral components that direct the genetic material to the nucleus and initiate transcription, and the ability to grow and purify high titers of the vector. However, the utilization of viruses alone as an in vivo delivery system for the induction of immune responses has limitations. One such limitation is the cellular tropism of the virus. Although some viruses are infective for a number of cell types, many virus types considered for gene therapy do not efficiently infect antigen presenting cells (APCs), such as dendritic cells (DCs). Infection of APCs is desirable for generating certain types of immune responses. An inability to infect DCs results in the need for cross priming for the induction of an immune response. Another limitation to the use of virus as a gene delivery system for vaccines is the possibility of immune responses being generated against the virus itself, as opposed to the encoded antigen. In some cases, neutralizing antibodies are produced against viral components that, once generated, significantly hinder the ability to use that vector to generate the desired immune response to the desired antigen. There remains a need for improved methods of delivering genetic material that provide the advantages of viral delivery while minimizing the disadvantages of using virus alone.

### SUMMARY OF THE INVENTION

The invention provides a viral vector conjugated to a microparticle, wherein the vital vector comprises a polynucleotide encoding a heterologous immunogenic polypeptide. Conjugation of the vital vector to the microparticle results in a dramatic increase in the efficacy of the elicited immune response. The viral vector can comprise a virus particle, a virus-like particle or a virus replicon particle. The microparticle can be conjugated to the viral vector by covalent interaction or by non-covalent interaction. In preferred embodiments, the vital vector is derived from a virus that enters cells via receptor mediated endocytosis, such as a rhinovirus, adenovirus, adeno-associated virus, enterovirus, poliovirus, coxsackie virus, echovirus, cardiovirus, hepatovirus, alphavirus, rubellavirus, flavivirus, pestivirus, hepatitis C virus, orthomyxovirus, bunyavirus, hantavirus, or nairovirus. In another embodiment, the viral vector is derived from a virus that enters cells via pH independent membrane fusion, such as a parainfluenza virus, mumps virus, measles virus or respiratory syncytial virus.

In some embodiments, the microparticle has a characteristic length of about 0.5 μm to about 20 μm. In a preferred embodiment, the microparticle comprises a positively charged surface. The positive charge may be due to characteristics of the wall-forming material itself, or to an additive that is added to the polymer-solvent solution and/or to the process media used in the preparation of the microparticles. The microparticle can further comprise a cationic lipid, a polymer of a natural or synthetic monomer, or an anionic surfactant. In some embodiments, the microparticle comprises polyvinyl alcohol, polyvinyl pyrilidone, carboxymethyl cellulose, gelatin, or polyoxyethylene(20) sorbitan monolaurate (e.g., Tween™ 20, Tween™ 80; Sigma-Aldrich Corp., St. Louis, Mo.).

The invention additionally provides a method for delivering a polynucleotide to a cell comprising contacting the cell with a microparticle of the invention. In a preferred embodiment, the cell is an antigen-presenting cell, such as a dendritic cell. The contacting can occur ex vivo or in vivo. In preferred embodiments, the heterologous polypeptide is an antigen or other immunogenic molecule. The antigen can be associated with cancer, autoimmune disease or infectious disease. In one embodiment, the antigen is associated with M. tuberculosis.

The invention further provides a vaccine comprising a microparticle of the invention and, optionally, further comprising an adjuvant. The invention thus provides a method for delivering a polynucleotide to a subject comprising administering to the subject a vaccine of the invention. Also provided is a method of stimulating an immune response in a subject, a method of treating cancer in a subject, a method of inhibiting tumor growth in a subject, a method of treating autoimmune disease in a subject, and a method of treating an infection in a subject.

### BRIEF DESCRIPTION OF THE FIGURES

FIG. 1 is a graph showing proliferation, as measured by incorporation of ³H-thymidine (in counts per minute, CPM), of 1-1B T cells in response to stimulation with dendritic cells that had been exposed to recombinant adenovirus encoding the M. tuberculosis antigen 38-1 (Adeno/38-1), Adeno/38-1 conjugated to microparticles of the invention (Adeno/38-1+Microparticles), Adeno/38-1 mixed with lipofectamine (Adeno/38-1+Lipofectamine), or Peptide 2-11, which is the minimal peptide recognized by 1-1B T cells.

FIG. 2 is a graph showing interferon gamma (IFN-γ) production, measured as optical density (O.D.) at 450-570 nm, by 1-1B-T cells in response to stimulation with dendritic cells that had been exposed to recombinant adenovirus encoding the M. tuberculosis antigen 38-1 (Adeno/38-1), Adeno/38-1 conjugated to microparticles of the invention (Adeno/38-1+Microparticles), Adeno/38-1 mixed with lipofectamine (Adeno/38-1+Lipofectamine), or Peptide 2-11, which is the minimal peptide recognized by 1-1B-T cells.

FIG. 3 is a graph showing interferon gamma (IFN-γ) production, measured as optical density (O.D.) at 450-570 nm, by 1-1B-T cells in response to stimulation with dendritic cells that had been exposed to recombinant adenovirus encoding the M. tuberculosis antigen TbH9 (TbH9 ad), microparticles made with polymer formulation A (TC339), TbH9 ad conjugated to formulation A microparticles (TbH9+TC339), recombinant adenovirus encoding the M. tuberculosis antigen 38-1 (38-1 ad), 38-1 ad conjugated to formulation A microparticles (38-1 ad+TC339), 38-1 ad conjugated to a second lot of formulation A microparticles (38-1 ad+TC350b), 38-1 ad conjugated to formulation C microparticles (38-1 ad+CD125), or 38-1 ad conjugated to formulation B microparticles (38-1 ad+CD165b).

FIG. 4 is a graph showing interferon gamma (IFN-γ) production, measured as optical density (O.D.) at 450-570 nm, by 1-1B-T cells in response to stimulation with dendritic cells that had been exposed to recombinant M. tuberculosis antigen 38-1 (r38-1), r38-1 conjugated to microparticles made with formulation A (r38-1+TC339), r38-1 ad conjugated to microparticles made with a second lot of formulation A (r38-1+TC350b), or r38-1 conjugated to formulation C microparticles (r38-1+CD125).

FIG. 5 is a graph showing interferon gamma (IFN-γ) production, measured as optical density (O.D.) at 450-570 nm and plotted as a function of multiplicity of infection (MOI), by DPV specific murine CD8+ T cells in response to stimulation with DC2.4 antigen presenting cells that had been infected 24 hours prior with either a recombinant DPV adenovirus or a control recombinant adenovirus (TbH9 adeno). The adenovirus was added naked, or following preincubation with lipfectamine (lipo) or was pre-adsorbed to PLG microspheres (ms). After 48 hours of culture, supernatants were collected for the assessment of IFN-γ production.

FIG. 6 is a graph showing T cell proliferation (CPM incorporated) as a function of multiplicity of infection (MOI) for cells prepared as described for FIG. 5, with the exception of the final step. After 48 hours in culture, the plates were pulsed with tritiated thymidine to measure the proliferation of T cells.

FIGS. 7A-C are photomicrographs showing that conjugation of adenovirus to cationic microparticles augments infection of human DC: Adenovirus expressing hrGFP was added to 2.5×10⁵ human DC alone (naked; 7A), treated with lipofectamine (7B) or conjugated to microparticle formulation A (7C) at an MOI of 20 and incubated for 16 hrs at 37° C. hrGFP expression was visualized by fluorescence microscopy at 10× magnification.

FIGS. 8A-D are photomicrographs showing that conjugation of adenovirus to cationic microparticles allows detectable expression at low MOI: Adenovirus expressing hrGFP was conjugated to formulation A and subjected to ⅕ serial dilution. The diluted conjugates were added to 2.5×10⁵ human DC per well at an MOI of 100 (8A), 20 (8B), 4 (8C) or 0.8 (8D) and incubated 16 hrs at 37° C. Expression was visualized by fluorescence microscopy at 10× magnification.

### DETAILED DESCRIPTION OF THE INVENTION

The invention is based on the discovery that delivery of combinations of viral vectors and microparticles facilitate the infection of phagocytic antigen-presenting cells (APCs). Data described herein show that adenovirus, which alone does not efficiently infect dendritic cells, does infect dendritic cells more effectively when combined with microparticles. These infected cells subsequently present the vitally encoded heterologous antigen to T cells in a productive manner. As a result, a far more effective immune response can be elicited when viral vectors encoding immunogenic polypeptides are conjugated to microparticles. This invention therefore provides compositions and methods for delivery of immunogenic molecules that offers the advantages of viral delivery systems while also overcoming problems encountered with delivery using virus alone.

Definitions

All scientific and technical terms used in this application have meanings commonly used in the art unless otherwise specified. As used in this application, the following words or phrases have the meanings specified.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally occurring nucleotides.

As used herein, “polypeptide” includes proteins, fragments of proteins, and peptides, whether isolated from natural sources, produced by recombinant techniques or chemically synthesized. Polypeptides of the invention typically comprise at least about 8 amino acids.

As used herein, “conjugated” refers to the joining together of elements, such as by covalent or non-covalent interactions. Examples of covalent interactions include chemical bonds; and examples of non-covalent interactions include ionic interactions, van der Waals forces, and hydrophobic and hydrophilic interactions. One example of conjugation of a viral vector to a microparticle is surface adsorption of the viral vector to the microparticle.

As used herein, “viral vector” means a virus particle, virus-like particle, virus replicon particle, or an equivalent thereof, that includes a polynucleotide encoding a polypeptide that is not native to the virus.

As used herein, an “immune response” is evidenced by conventional indicators of a protective immune response, including, but not limited to, release of gamma interferon (IFN-γ), T cell proliferation, and cytokine or antibody production.

As used herein, “microparticle” refers to a material comprising a wall forming material and having surface charge characteristics, size and morphology capable of delivering a vital vector into a cell. Microparticles may be solid or porous, have a rough or smooth surface, and may have a regular or irregular shape. Examples of microparticles include, but are not limited to, microspheres, sheets, rods and tubes.

As used herein, “characteristic length” means length, width or diameter of a microparticle, as appropriate given the morphology of the relevant microparticle.

As used herein, “subject” refers to the recipient of the therapy to be practiced according to the invention. The subject can be any vertebrate, but will preferably be a mammal. If a mammal, the subject will preferably be a human, but may also be a domestic livestock, laboratory subject or pet animal.

As used herein, to “prevent” a disease or condition means to hinder or delay the onset or development of the disease or condition.

As used herein, to “treat” a disease or condition means to ameliorate one or more symptoms associated with the disease or condition.

As used herein, “antigen-presenting cell” or “APC” means a cell capable of handling and presenting antigen to a lymphocyte. Examples of APCs include, but are not limited to, macrophages, Langerhans-dendritic cells, follicular dendritic cells, B cells, monocytes, fibroblasts and fibrocytes. Dendritic cells are a preferred type of antigen presenting cell. Dendritic cells are found in many non-lymphoid tissues but can migrate via the afferent lymph or the blood stream to the T-dependent areas of lymphoid organs. In non-lymphoid organs, dendritic cells include Langerhans cells and interstitial dendritic cells. In the lymph and blood, they include afferent lymph veiled cells and blood dendritic cells, respectively. In lymphoid organs, they include lymphoid dendritic cells and interdigitating cells.

As used herein, “modified” to present an epitope refers to antigen-presenting cells (APCs) that have been manipulated to present an epitope by natural or recombinant methods. For example, the APCs can be modified by exposure to the isolated antigen, alone or as part of a mixture, peptide loading, or by genetically modifying the APC to express a polypeptide that includes one or more epitopes.

As used herein, “pharmaceutically acceptable salt” refers to a salt that retains the desired biological activity of the parent compound and does not impart any undesired toxicological effects. Examples of such salts include, but are not limited to, (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, furmaric acid, gluconic acid, citric acid, malic acid, ascorbic acid, benzoic acid, tannic acid, pamoic acid, alginic acid, polyglutamic acid, naphthalenesulfonic acids, naphthlalenedisulfonic acids, polygalacturonic acid; (b) salts with polyvalent metal cations such as zinc, calcium, bismuth, barium, magnesium, aluminum, copper, cobalt, nickel, cadmium, and the like; or (c) salts formed with an organic cation formed from N,N′-dibenzylethylenediamine or ethylenediamine; or (d) combinations of (a) and (b) or (c), e.g., a zinc tannate salt; and the like. The preferred acid addition salts are the trifluoroacetate salt and the acetate salt.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.90%) saline.

Compositions comprising such carriers are formulated by well known conventional methods (see, for example, Remington's Pharmaceutical Sciences, Chapter 43, 14th Ed., Mack Publishing Co, Easton Pa. 18042, USA).

As used herein, “adjuvant” includes those adjuvants commonly used in the art to facilitate the stimulation of an immune response. Examples of adjuvants include, but are not limited to, helper peptide; aluminum salts such as aluminum hydroxide gel (alum) or aluminum phosphate; Freund's Incomplete Adjuvant and Complete Adjuvant (Difco Laboratories, Detroit, Mich.); Merck Adjuvant 65 (Merck and Company, Inc., Rahway, N.J.); AS-2 (Smith-Kline Beecham); QS-21 (Aquilla); MPL™ immunostimulant or 3d-MPL (Corixa Corporation); LEIF; salts of calcium, iron or zinc; an insoluble suspension of acylated tyrosine; acylated sugars; cationically or anionically derivatized polysaccharides; polyphosphazenes; aminoalkyl glucosaminide phosphate (ACP); isotucaresol; monophosphoryl lipid A and quil A; muramyl tripeptide phosphatidyl ethanolamine or an imunostimulating complex, including cytokines (e.g., GM-CSF or interleukin-2, -7 or -12) and immunostimulatory DNA sequences. In some embodiments, such as with the use of a polynucleotide vaccine, an adjuvant such as a helper peptide or cytokine can be provided via a polynucleotide encoding the adjuvant.

As used herein, “a” or “an” means at least one, unless clearly indicated otherwise.

Microparticles and Nucleic Acid Delivery Systems

The invention provides a polynucleotide delivery system comprising one or more vectors conjugated to a microparticle. Preferably, the vector is a vital vector, such as a virus particle, a virus-like particle, a virus replicon particle or an equivalent of any one of the foregoing. The vector comprises a polynucleotide encoding a heterologous, immunogenic polypeptide that is capable of eliciting or enhancing an immune response.

Microparticle morphology can include spheres, sheets, rods, tubes and other shapes, and be solid or porous. The microparticles can have smooth surfaces, angular surfaces, rough surfaces, porous surfaces, or sharp edges. Microparticle size can vary over a fairly broad range, e.g., from about 0.2 μm to about 40 μm in diameter or length, and still be effective. In one embodiment, the microparticles are about 0.5 μm to about 20 μm in diameter or length. Preferably, the microparticle diameter or length is about 1 to about 10 μm. Microparticles in this size range are well-suited to be phagocytosed by antigen-presenting cells, leading to effective T cell stimulation.

The microparticle material can comprise any of a wide range of particles, including such exemplary wall forming materials as described in U.S. Pat. No. 5,407,609. Biocompatible materials are preferred for uses that involve administration to patients. Biodegradable materials are also preferred.

Preferred are biodegradable polymers, such as poly(lacto-co-glycolide) (PLG), poly(lactide), poly(glycolide), poly(caprolactone), poly(hydroxybutyrate) and/or copolymers thereof. Alternatively, the microparticles can comprise another wall-forming material. Suitable wall-forming materials include, but are not limited to, poly(dienes) such as poly(butadiene) and the like; poly(alkenes) such as polyethylene, polypropylene, and the like; poly(acrylics) such as poly(acrylic acid) and the like; poly(methacrylics) such as poly(methyl methacrylate), poly(hydroxyethyl methacrylate), and the like; poly(vinyl ethers); poly(vinyl alcohols); poly(vinyl ketones); poly(vinyl halides) such as poly(vinyl chloride) and the like; poly(vinyl nitriles), poly(vinyl esters) such as poly(vinyl acetate) and the like; poly(vinyl pyridines) such as poly(²-vinyl pyridine), poly(5-methyl-2-vinyl pyridine) and the like; poly(carbonates); poly(esters); poly(orthoesters); poly(esteramides); poly(anhydrides); poly(urethanes); poly(amides); cellulose ethers such as methyl cellulose, hydroxyethyl cellulose, hydroxypropyl methyl cellulose, and the like; cellulose esters such as cellulose acetate, cellulose acetate phthalate, cellulose acetate butyrate, and the like; poly(saccharides), proteins, gelatin, starch, gums, resins, and the like. These materials may be used alone, as physical mixtures (blends), or as copolymers.

Biodegradable microspheres (e.g., polylactate polyglycolate) for use as carriers are disclosed, for example, in U.S. Pat. Nos. 4,897,268; 5,075,109; 5,928,647; 5,811,128; 5,820,883; 5,853,763; 5,814,344; 5,407,609; and 5,942,252; the disclosures of each of which are incorporated herein by reference. In particular, these patents, such as U.S. Pat. Nos. 4,897,268 and 5,407,609, describe the production of biodegradable microspheres for a variety of uses, but do not teach the optimization of microsphere formulation and characteristics for DNA delivery.

Microparticle Formulation

The invention provides a method for producing a microparticle, as well as a method for producing a viral vector conjugated to a microparticle. The method for producing a microparticle comprises dissolving a polymer in a solvent to form a polymer solution. Optionally, a cationic compound, such as DOTAP, can be added to the solvent. The polymer solution is then emulsified and diluted. After mixing, the microparticles are hardened. The method can further comprise subsequent steps of washing, freezing and lyophilizing the microparticles. Alternatively, a double-emulsion technique can be used.

In some embodiments, the microparticle comprises a cationic lipid, a polymer of a natural or synthetic monomer, or an anionic surfactant. In a preferred embodiment, the microparticle comprises a positively charged surface. Both cationic and anionic components can be introduced into the microparticles to manipulate surface charge. A positive surface charge, for example, can be created by selection of a wall-forming material that will impart positive charges on the surface, such as polylysine, modified PLG, for example. Alternatively, additives, such as DOTAP or other charged compounds, can be added to the polymer-solvent solution and/or to the process media, to alter the surface charge of the resulting microparticles.

In a preferred embodiment, the polymer comprises PLG. In some embodiments, the PLG can include ester end groups or carboxylic acid end groups, and have a molecular weight of from about 4 kDa to about 120 kDa, or preferably, about 8 kDa to about 65 kDa. The solvent can comprise, for example, dichloromethane, chloroform, or ethylacetate. In some embodiments, the polymer solution further comprises a cationic lipid and/or an adjuvant, such as MPL. Examples of stabilizers include, but are not limited to, carboxymethylcellulose (CMC), polyvinyl alcohol (PVA), polyvinyl pyrrolidone (PVP), or a mixture thereof. The stabilizer can optionally further comprise a cationic lipid. In some embodiments, the stabilizer comprises from about 0 to about 10% of the process medium, or preferably, about 1% to about 5% of the process medium. In some embodiments, the solvent comprises an internal water volume of from about 0.001% to about 0.5%; and/or the aqueous solution comprises an ethanol content of from about 0% to about 75% (v/v).

The solvent used to dissolve the wall material or excipient can be selected from a variety of common organic solvents including halogenated aliphatic hydrocarbons such as methylene chloride, chloroform, and the like; alcohols; aromatic hydrocarbons such as toluene and the like; halogenated aromatic hydrocarbons; ethers such as methyl t-butyl ether and the like; cyclic ethers such as tetrahydrofuran and the like; ethyl acetate; diethyl carbonate; acetone; cyclohexane; and water. These solvents may be used alone or in combination. The solvent chosen must be a material that will dissolve the wall material or excipient and it is best that it is chemically inert with respect to the polymer or other components of the viral delivery system. Moreover, the solvent should have limited solubility in the extraction medium. Generally, limited solubility means having a solubility from about 1 part per 100 to about 25 parts per 100. Preferred solvents include ethyl acetate, diethyl carbonate, chloroform and methylene chloride.

Viral Vectors

The viral vector can comprise a virus particle, a virus-like particle or a virus replicon particle. Viruses, virus-like particles and virus replicon particles that normally enter a cell via receptor mediated endocytosis are particularly well-suited to this delivery system due to their increased rate and efficiency of APC infection as well as their enhanced antigen presentation over virus alone. This includes a large variety of both enveloped and non-enveloped viruses. It is also likely that infection of phagocytic cells by some viruses that normally enter cells via other mechanisms, such as pH independent membrane fusion, will also be aided by conjugation with microparticles.

The list of viruses that may be used for vaccine delivery in this microparticle-virus system is very broad, encompassing any variety of viruses. In one embodiment, the virus is selected from those viruses that normally enter a cell by receptor mediated endocytosis. In another embodiment, the virus is selected from those viruses which have already been developed for expression of heterologous recombinant genes. Examples of viruses that enter cells via receptor mediated endocytosis include, but are not limited to, rhinovirus, adenovirus, adeno-associated virus, enterovirus, poliovirus, coxsackie virus, echovirus, cardiovirus, hepatovirus, alphavirus, rubellavirus, flavivirus, pestivirus, hepatitis C virus,, orthomyxovirus, bunyavirus, hantavirus, or nairovirus. Examples of viruses that enter cell, via pH independent membrane fusion include, but are not limited to, parainfluenza virus, mumps virus, measles virus, respiratory syncytial virus, retrovirus, herpes virus and pox virus.

Microparticles and viruses can be combined for in vivo or in vitro use in a variety of manners including, but not limited to, pre-, post-lyophilization, solution/dry, buffer type and pH, excipients present, time and temperature of incubation, mixing conditions, ratios of particles and virus, and concentrations of particles and virus. The microparticle can be conjugated to the vital vector by covalent interaction or by non-covalent interaction. Examples of covalent interactions include chemical bonds; and examples of non-covalent interactions include ionic interactions, van der Waals forces, and hydrophobic and hydrophilic interactions. One example of conjugation of a viral vector to a microparticle is surface adsorption of the viral vector to the microparticle.

Compositions

The invention provides compositions that are useful for delivering polynucleotides. In one embodiment, the composition is a pharmaceutical composition. The composition can comprise a therapeutically or prophylactically effective amount of a polynucleotide that encodes an immunogenic polypeptide. An effective amount is an amount sufficient to elicit or augment an immune response, e.g., by activating T cells. One measure of the activation of T cells is a cytotoxicity assay or an interferon-gamma release assay, as described in the examples below. In some embodiments, the composition is a vaccine.

In some embodiments, the condition to be treated or prevented is cancer or a precancerous condition (e.g., hyperplasia, metaplasia, dysplasia). Examples of cancer include, but are not limited to, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oliodendroglioma, meningioma, melanoma, neuroblastoma, tetinoblastoma, leukemia, lymphoma, multiple myeloma, Waldenström's macroglobulinemia, and heavy chain disease.

In some embodiments, the condition to be treated or prevented is an infectious disease. Examples of infectious disease include, but are not limited to, infection with a pathogen, virus, bacterium, fungus or parasite. Examples of viruses include, but are not limited to, hepatitis type B or type C, influenza, varicella, adenovirus, herpes simplex virus type I or type II, rinderpest, rhinovirus, echovirus, rotavirus, respiratory syncytial virus, papilloma virus, papova virus, cytomegalovirus, echinovirus, arbovirus, hantavirus, coxsachie virus, mumps virus, measles virus, rubella virus, polio virus, human immunodeficiency virus type I or type II. Examples of bacteria include, but are not limited to, M. tuberculosis, mycobacterium, mycoplasma, neisseria and legionella. Examples of parasites include, but are not limited to, rickettsia and chlamydia.

The composition can optionally include a carrier, such as a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention. Formulations suitable for parenteral administration, such as, for example, by intraarticular (in the joints), intravenous, intramuscular, intradermal, intraperitoneal, and subcutaneous routes, and carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, liposomes, microspheres and emulsions.

The composition of the invention can further comprise one or mote adjuvants. Examples of adjuvants include, but are not limited to, helper peptide, alum, Freund's, muramyl tripeptide phosphatidyl ethanolamine or an immunostimulating complex, including cytokines. In some embodiments, such as with the use of a polynucleotide vaccine, an adjuvant such as a helper peptide or cytokine can be provided via a polynucleotide encoding the adjuvant. A preferred adjuvant is AGP.

Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); aluminum salts such as aluminum hydroxide gel (alum) or aluminum phosphate; salts of calcium, iron or zinc; an insoluble suspension of acylated tyrosine acylated sugars; cationically or anionically derivatized polysaccharides; polyphosphazenes biodegradable microspheres; monophosphoryl lipid A and quil A. Cytokines, such as GM CSF or interleukin-2, -7, or -12, may also be used as adjuvants.

Within the vaccines provided herein, the adjuvant composition is preferably designed to induce an immune response predominantly of the Th1 type. High levels of Th1-type cytokines (e.g., IFN-γ, IL-2 and IL-12) tend to favor the induction of cell mediated immune responses to an administered antigen. In contrast, high levels of Th2-type cytokines (e.g., IL-4, IL-5, IL-6, IL-10 and TNF-β) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, 1989, Ann. Rev. Immunol. 7:145-173.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corporation (Hamilton, Mont.) (see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555. Another preferred adjuvant is a saponin, preferably QS21, which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739.

Other preferred formulations comprise an oil-in-water emulsion and tocopherol. A particularly potent adjuvant formulation involving QS21, 3D -MPL and tocopherol in an oil-in-water emulsion is described in WO 95/17210. Another adjuvant that may be used is AS-2 (Smith-Kline Beecham). Any vaccine provided herein may be prepared using well known methods that result in a combination of antigen, immune response enhancer and a suitable carrier or excipient.

Vaccine preparation is generally described in, for example, M. F. Powell and M. J. Newman, eds., “Vaccine Design (the subunit and adjuvant approach),” Plenum Press (NY, 1995). Pharmaceutical compositions and vaccines within the scope of the present invention may also contain other compounds, which may be biologically active or inactive.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes using well known technology.

The compositions described herein may be administered as part of a sustained release formulation (i.e., a formulation such as a capsule or sponge that effects a slow release of compound following administration). Such formulations may generally be prepared using well known technology and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

Methods

The invention provides a method for delivering a polynucleotide to a cell. The method comprises contacting the cell with a polynucleotide delivery system of the invention, such as a viral vector conjugated to a microparticle. The contacting can occur ex vivo or in vivo. The cell can be a patient's own cells, to be re-introduced to the patient after ex vivo contact with the delivery system or to be contacted in vivo following administration of the delivery system to the patient. In addition, the contacting can occur in an ex vivo environment for any purpose in which polynucleotide delivery into a cell is desired. In one embodiment, the cell is an antigen-presenting cell, such as a dendritic cell. In other embodiments, other cells, capable of expressing the polynucleotide are used.

Likewise, the invention provides a method for delivering a polynucleotide to a subject. The method comprises administering to the subject a polynucleotide delivery system, or a composition, of the invention. The invention further provides a method of stimulating an immune response to an immunogenic polypeptide in a subject, a method of inhibiting tumor growth in a subject, a method of prolonging survival in a subject having a cancer, and a method of treating or preventing cancer, autoimmune disease, or infectious disease. The method comprises administering to the subject a composition or delivery system of the invention.

Administration of the Compositions

Treatment includes prophylaxis and therapy. Prophylaxis or treatment can be accomplished by a single direct injection at a single time point or multiple time points. Adminstration can also be nearly simultaneous to multiple sites. Patients or subjects include mammals, such as human, bovine, equine, canine, feline, porcine, and ovine animals. Preferably, the patients or subjects are human.

Compositions are typically administered in vivo via parenteral (e.g. intravenous, subcutaneous, and intramuscular) or other traditional direct routes, such as buccal/sublingual, rectal, oral, nasal, topical, (such as transdermal and ophthalmic), vaginal, pulmonary, intraarterial, intraperitoneal, intraocular, or intranasal routes or directly into a specific tissue.

The dose administered to a patient, in the context of the present invention should be sufficient to effect a beneficial therapeutic response in the patient over time, or to inhibit infection or disease due to infection. Thus, the composition is administered to a patient in an amount sufficient to elicit an effective immune response to the specific antigens and/or to alleviate, reduce, cure or at least partially arrest symptoms and/or complications from the disease or infection. An amount adequate to accomplish this is defined as a “therapeutically effective dose.”

The dose will be determined by the activity of the composition produced and the condition of the patient, as well as the body weight or surface areas of the patient to be treated. The size of the dose also will be determined by the existence, nature, and extent of any adverse side effects that accompany the administration of a particular composition in a particular patient. In determining the effective amount of the composition to be administered in the treatment or prophylaxis of diseases, the physician needs to evaluate the production of an immune response against the pathogen, progression of the disease, and any treatment-related toxicity.

Administration by many of the routes of administration described herein or otherwise known in the art may be accomplished simply by direct administration using a needle, catheter or related device, at a single time point or at multiple time points.

### EXAMPLES

The following examples are presented to illustrate the present invention and to assist one of ordinary skill in making and using the same. The examples are not intended in any way to otherwise limit the scope of the invention.

### Example 1

### Microparticle Preparation

Microparticles were prepared using a variation on the double emulsion technique. Poly(lactide-co-glycolide) (PLG) polymer (MW˜40,000 Da) having ester end groups was dissolved in a solvent, dichloromethane, to a concentration of 33 mg/ml. 25 mg of a cationic compound, dioleoyl-1,2-diacyl-3-trimethylammonium-propane (DOTAP), was also added to the solvent. This mixture was then emulsified in 15 ml of an aqueous solution containing 5% polyvinyl alcohol (PVA) using a Silverson mixer. The dispersion was then diluted with 50 ml of 1% PVA and mixed for several hours. The hardened microspheres were washed several times with distilled water, collected by centrifugation, and lyophilized in the presence of mannitol. Particles were prepared in the absence of viral particles, and the end product was suitable for subsequently being combined with the viral material. This microparticle formulation is referred to herein as “Formulation A”, and includes two lots, identified as TC339 and TC350b.

Additional variations on the microparticle formulation have also been prepared. Formulation B is prepared in manner similar to that described above for Formulation A, except that the polymer used in formulation B has a mean molecular weight of about 8-10 kDa and has acid end groups (rather than ester end groups). Two lots of formulation B are referred to herein as CD056 and CD150b.

A third microparticle formulation, referred to as “Formulation C”, has also been prepared. Formulation C was prepared with PLG having an average molecular weight of about 40,000 Da and ester end groups, and dissolved in dichloromethane (DCM; 86 mg/ml). 5.1 ml of aqueous solution was emulsified into this solution using a Polytron mixer for 20 seconds. This primary emulsion was added to 280 ml of an aqueous solution of CMC (1.4%) and emulsified at 4500 for 75 seconds using a Silverson mixer. The microparticles were mixed for several hours.

### Example 2

### Microparticle Characterization

A Horiba LA920 particle size analyzer was used to determine the size and size distribution of the microparticles. In addition, an aliquot was set aside for examination using scanning electron micrographs (SEM). The surface charge (zeta-potential) of the microparticles was measured using a Malvern Zetasizer. The microparticles were also assayed for their ability to bind plasmid DNA to their surface. Briefly, a solution of plasmid DNA (3-10 kbp) was used to disperse the microparticles. After incubation, the microparticles were centrifuged, and the supernatant removed for analysis. DNA concentrations were quantified using the Picogreen DNA assay (Molecular Probes, Eugene, Oreg.). The difference between the DNA concentration of the supernatant and that of the initial DNA solution was used to determine the amount of DNA which adsorbed to the microparticles.

Scanning electron microscopy revealed that these three microparticle formulations (A, B and C) resulted in microparticles that were predominantly spherical in shape with little to no surface porosity. Particle size analysis indicated that the median particle diameters for the microparticles of these lots was between ˜0.5 and 10 μm. The zeta-potentials was determined to be −4 mV, +27 mV and −29 mV for formulations A, B and C, respectively.

Addition of the cationic additive in formulation A did not yield a net positive zeta-potential, but did yield microparticles with a greater net surface charge than those lots prepared in similar manners but without cationic additives (i.e., formulation C). Interestingly, replacing the type of PLG in formulation A (higher molecular weight with ester end groups) with PLG having carboxylic acid end groups and a lower molecular weight (formulation B) produced a significant difference in the surface charge: +27 mV for B versus −4 mV for A. As an additional measure of the surface properties of these formulations, the ability of plasmid DNA to bind to their surfaces was also quantified. The ability of these formulations to bind DNA, shown in Table 1, was found to vary with the most cationic formulation, B, binding the most DNA, and formulation A binding little DNA. Immunological studies show that formulation B is not as attractive as A, suggesting that a balanced level of cationic surface charges is best. This may indicate that a zwitterionic surface is best.

TABLE 1
DNA Adsorption to Select Lots of Microspheres Prepared with a
Cationic Component

| | Input | | DNA Adsorbed | | |
|---|---|---|---|---|---|
| Formulation | DNA | Formulation | Amount | | Loading |
| Description | (μg) | (mg) | (μg) | (%) | (w/w) |
| CD056 | 50 | 5.0 | 26.9 | 54% | 0.54% |
| TC339 | 49 | 7.2 | 4.6 | 9% | 0.06% |
| Control (naked DNA) | 50 | 0.0 | 1.4 | 3% | — |
| Control (naked DNA) | 155 | 0.0 | 4.6 | 3% | — |
| Control (Microparticles | 0 | 5.7 | 0 | — | 0.00% |
| alone) | | | | | |

Table 1 shows that DNA was able to effectively adsorb to lot CD056 (Formulation B), with 26.9 μg out of the initial 50 μg input being adsorbed to the particles. In contrast, little DNA was adsorbed to the microparticles utilized in the experiment described below (Formulation A). Table 1 suggests that a small amount of DNA may have adsorbed to TC339 (4.6 μg). It is interesting to note that formulations CD056 (B) and TC339) (A) were prepared in identical manners with the sole exception of the polymer used. Hence, the ability of plasmid DNA to bind is influenced by more subtle factors than simply the presence or absence of a cationic component.

### Example 3

### Preparation of Virus

Adenoviral vectors expressing TB antigens 38-1, DPV, and TbH9 were made by subcloning the reading frame of the antigen into pShuttle CMV. The resultant plasmid, pShuttle CMV/38-1 was recombined with pAdEasy1 in E. coli BJ5183 cells. The recombination event between the two plasmids assembled a chimeric plasmid, pAd38-1 containing the complete adenovirus setotype 5 genome with the expression cassette replacing the viral E1 region. The bacterial portion of pAd38-1 was removed by PacI digestion and this DNA was used to transfect human embryonic kidney 293 cells (HEK293) to generate a recombinant adenoviral vector. The virus stock was purified over CsCl gradients and dialyzed against Hepes buffered saline. The biological titer was determined by plating dilutions of the purified virus on HEI(293 cells.

### Example 4

### Functional Assay for Presentation of Virally Encoded Antigen

1-1B is a class I restricted (HLA-B44) CD8⁺ T-cell clone that is specific for the Mycobacterium Tuberculosis antigen 38-1 (also known as CFP-10/Mtb11). 20,000 1-1B-T cells were cultured with HLA-matched monocyte-derived dendritic cells (DC, 20,000/well) that had been infected 24 hr before with recombinant adenovirus expressing the 38-1 gene or other control, as described below. Cultures were performed in triplicate in flat-bottomed 96-well microtiter plates. The test conditions for treatment of dendritic cells included: (1) 38-1 adenovirus alone (adeno/38-1); (2) adeno/38.1 plus microparticles after 30 minutes incubation at room temperature; (3) adeno/38.1 mixed with lipofectamine prior to addition to DC; and (4) peptide 2-11, which is the minimal peptide recognized by these T cells.

The DCs were cultured with these stimuli for 2 hr, at which time the supernatant was removed and the cells were washed once with culture medium. The medium was replaced and the cells were cultured overnight in fresh medium containing GM-CSF and IL-4 (20 ng/ml of each). This medium was removed and replaced with medium containing 1-1B-T cells to give a final volume of 200 μl/well. The plates were cultured for an additional 72 hr, when 50 μ of supernatant was removed for determination of IFN-γ levels by ELISA. The plates were then pulsed overnight with tritiated thymidine (1 μCi/well).

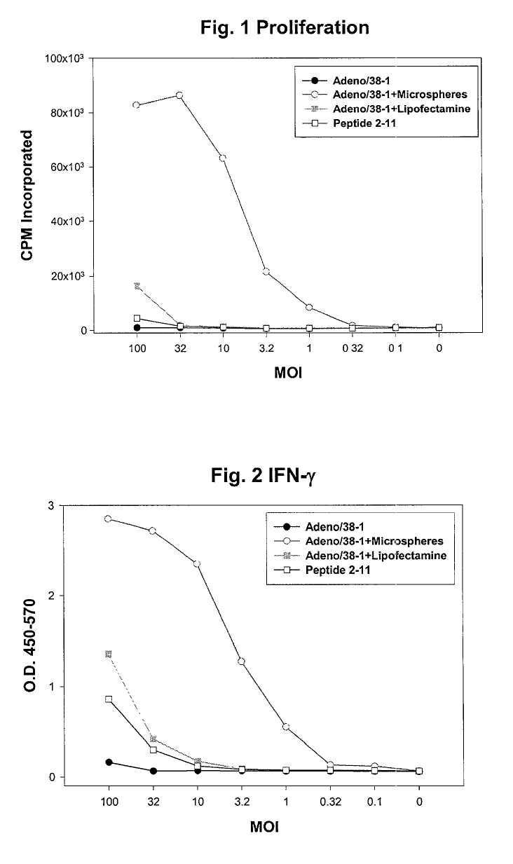

A CD8+ CTL clone termed 1-1B that responds specifically to the Mycobacterium tuberculosis antigen 38-1 was utilized to compare the presentation of the virally encoded antigen generated by the microparticle-adenovirus formulation to that of adenovirus particles alone. Adenovirus plus Lipofectamine was included as a positive control, as this combination has previously been shown to produce increased levels of infection over virus alone in in vitro systems. FIG. 1 shows the proliferation (thymidine incorporation) responses in this in vitro assay. FIG. 1 demonstrates that adenovirus alone was inefficient at stimulating 1-1B cells. The use of Lipofectamine marginally improved the stimulation of 1-1B T cells. In contrast, the microparticle-adenovirus formulation strongly enhanced stimulation of 1-1B T cells such that a multiplicity of infection (MOI) of 1 was more stimulatory than seen with an MOI of 100 with the naked 38-1 adenovirus (FIG. 1). Similarly, for IFN-γ production approximately a 300-fold greater MOI was needed to stimulate the 1-1B-T cells with naked 38-1 adenovirus compared with adenovirus adsorbed to microspheres (FIG. 2).

### Example 5

### Additional Microparticle Formulations Provide Effective Vehicles for Viral Delivery

Multiple formulations may successfully adsorb virus and, likely, some will be more efficient than others. To investigate this possibility, additional microparticle formulations were utilized in a subsequent experiment conducted in the same manner previously described, with the exception that media was exchanged after 24 hours rather than 2 hrs. In this experiment, the formulation used in FIG. 1, MPAdVTC339/38-1 (using the TC339 microparticle lot of Formulation A) was compared to a second lot of an identical formulation in addition to a cationic microparticle formulation utilizing a different polymer (CD165b) and a non-cationic microparticle.

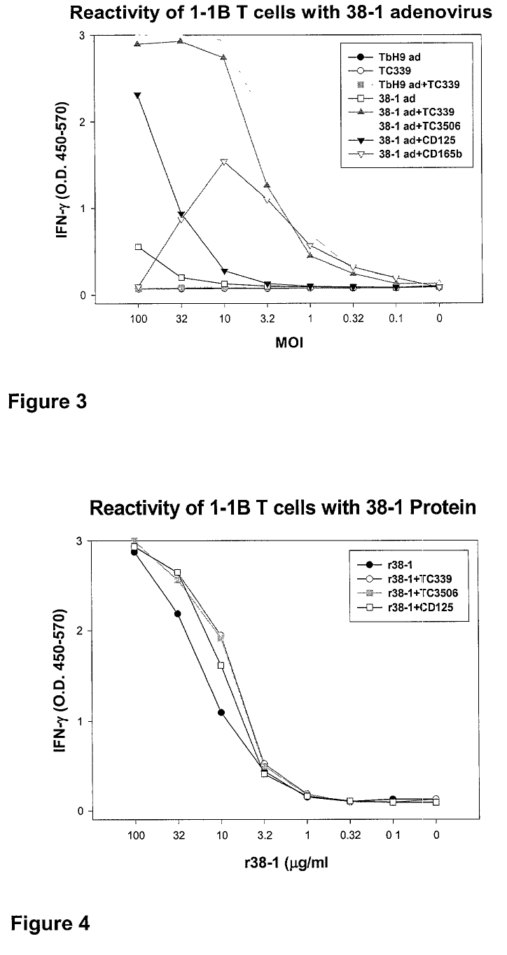

As shown in FIG. 3, the TC339 and TC350b give identical results, indicating that the preparation of this microparticle formulation (A) is reproducible. These results also indicate that, while an additional cationic microparticle formulation (CD 165b, Formulation B) is capable of increasing infection and antigen presentation of the DC, it decreases the efficiency of presentation at higher concentrations. There are several possible explanations for this effect, including cell toxicity and steric hindrance of the cellular interaction. The non-cationic microparticles (CD125, Formulation C) are also capable of enhancing antigen presentation of the Adv-encoded antigen, however, they do so at an efficiency approximately 30-fold lower than TC339 (Formulation A). The control AdV-38-1 alone was again quite inefficient at infecting the DC, resulting in significant levels of antigen presentation only at an MOI approaching 100:1. The TC339 microparticles alone gave no T cell activation, as did an AdV construct coding for a different TB antigen, whether alone or conjugated to microparticle formulation TC339.

The results shown in FIG. 4 indicate that these formulations contribute only marginally to DC antigen presentation when mixed with 38-1 recombinant protein. While exogenous protein alone can be processed and presented, very high quantities (100 μg/ml) of protein are required to achieve comparable activation to that obtained with the TC339 MPAdV formulation.

### Example 6

### Presentation of Virally Encoded Antigen Delivered as Conjugates with Microparticles is Effective for Multiple Antigens

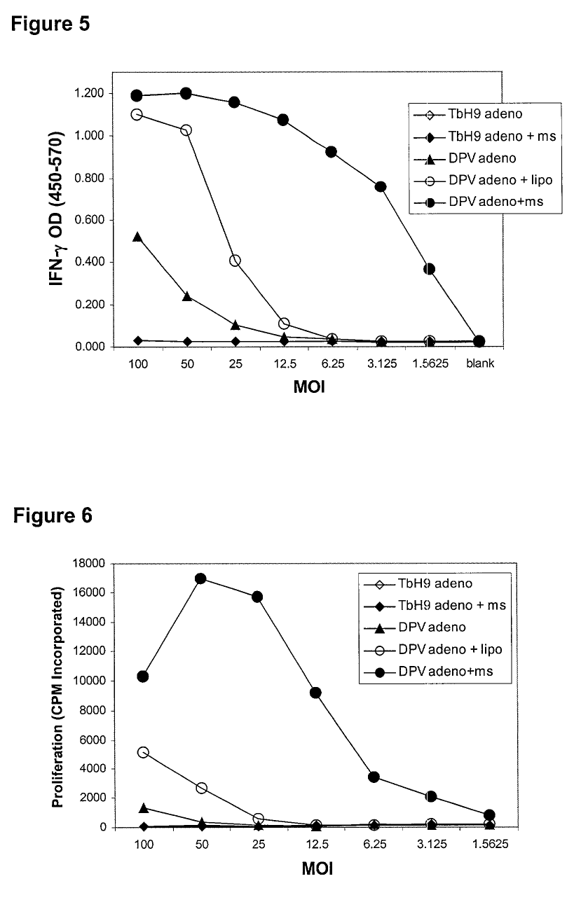

To demonstrate that the stimulatory effect of the adenovirus infected DC can be achieved with multiple antigens, experiments were conducted identical to those described in Example 5 above, utilizing a CD8+ T cell clone specific for a different TB antigen, DPV. As an additional control, DC were infected with a third TB antigen, TbH9, to demonstrate non-reactivity to an irrelevant antigen. As shown in FIGS. 5 and 6, DC infected with naked adenovirus expressing the DPV antigen only marginally activate the DPV specific CD8+ T cell. Conjugation of the adenoviral vector to microparticles again results in dramatic improvement of antigen presentation for both IFNγ production (FIG. 5) and proliferation (FIG. 6). DC infected with the irrelevant TB antigen, TbH9, showed no activation of the DPV-specific T cell clone in either assay.

### Example 7

### Improved Infection of Human Dendritic Cells by Adenovirus Conjugated to Microspheres

This example demonstrates dramatic improvement of in vitro infection of human dendritic cells by utilizing adenovirus conjugated to cationic microspheres. Recombinant adenovirus expressing hrGFP (Stratagene) was conjugated to microparticle formulation A (Example 1). Recombinant adenovirus conjugated to microparticles was compared to naked adenovirus and virus pre-incubated with lipofectamine. Virus conjugates were plated with human DC at various MOI for 16 hours at 37° C. Gene expression was monitored by fluorescence microscopy.

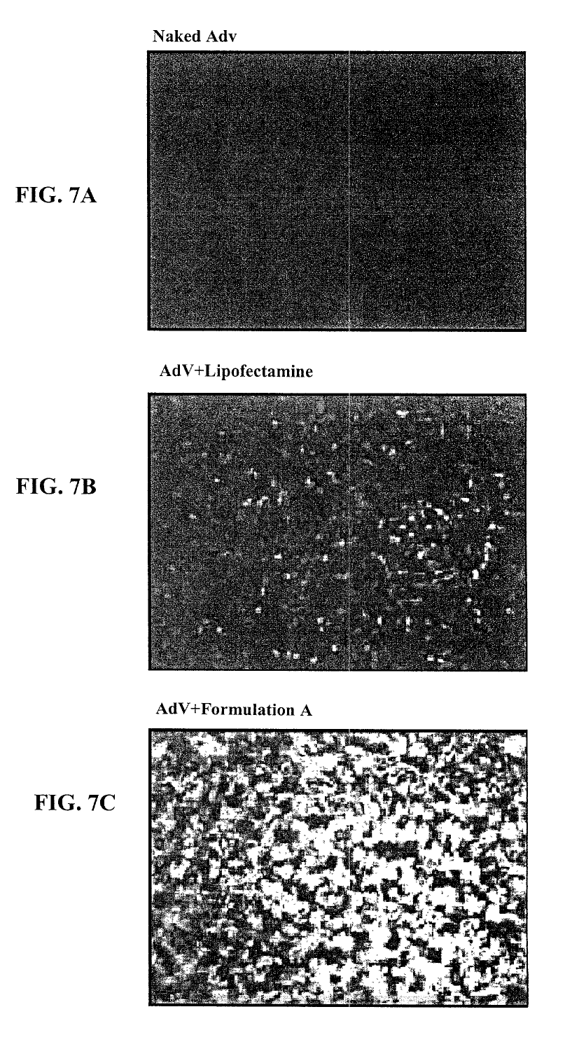

As shown in FIG. 7A, naked adenovirus does not infect human DC efficiently at an MOI of 20. Pre-incubation of the virus particles with lipofectamine greatly improves infection at this time point (FIG. 7B). However, conjugation of the adenovirus particles with cationic microspheres shows a dramatic increase in infectivity, not only over naked adenovirus, but also over the virus/lipofectamine combination (FIG. 7C).

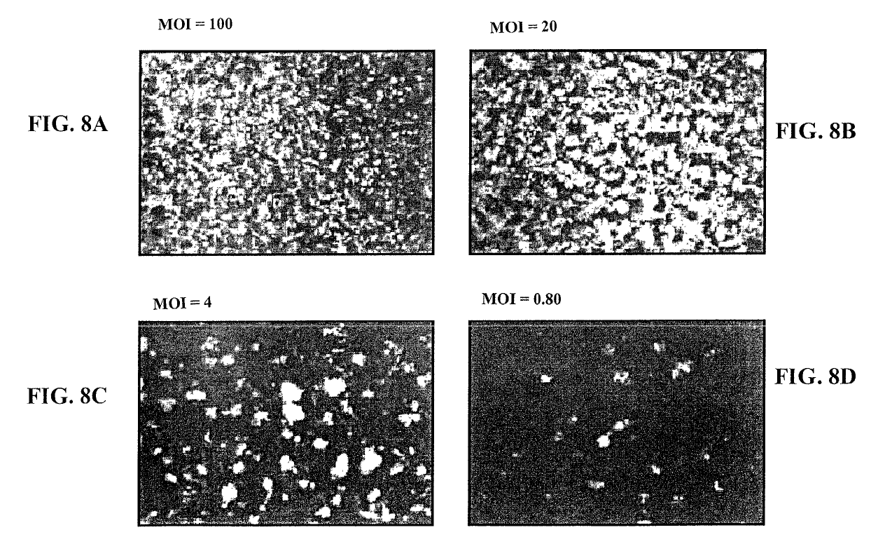

To determine the extent to which the microparticle conjugates improve infection, titrations of the conjugates were incubated with the dendritic cells. While gene expression in the cells exposed to naked adenovirus is barely detectable after 16 hr at an MOI of 20 (FIG. 7A), the adenovirus/microparticle conjugates show clear gene expression at an MOI as low as 0.8 (FIGS. 8A-D). Thus, conjugation of recombinant adenovirus containing transgene to microparticles of the invention dramatically improves the efficiency of human dendritic cell infection.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A viral vector conjugated to a microparticle, wherein the viral vector comprises a polynucleotide encoding a heterologous immunogenic polypeptide.

2. The viral vector of claim 1, which comprises a virus particle, a virus-like particle or a virus replicon particle.

3. The viral vector of claim 1, which is derived from a virus that enters cells via receptor mediated endocytosis.

4. The viral vector of claim 3, wherein the virus is a rhinovirus, adenovirus, enterovirus, poliovirus, coxsackie virus, echovirus, cardiovirus, hepatovirus, alphavirus, rubellavirus, flavivirus, pestivirus, hepatitis C virus, orthomyxovirus, bunyavirus, hantavirus, or nairovirus.

5. The viral vector of claim 1, wherein the viral vector is derived from a virus that enters cells via pH independent membrane fusion.

6. The viral vector of claim 5, wherein the virus is a parainfluenza virus, mumps virus, measles virus, respiratory syncytial virus, retrovirus, herpes virus or pox virus.

7. The viral vector of claim 1, wherein the microparticle has a characteristic length of about 0.5 μm to about 20 μm.

8. The viral vector of claim 1, wherein the microparticle comprises a wall-forming material selected from the group consisting of poly(lacto-co-glycolide) (PLG), poly(lactide), poly(glycolide), poly(caprolactone), poly(hydroxybutyrate) and copolymers thereof.

9. The viral vector of claim 1, wherein the microparticle comprises a positively charged surface.

10. The viral vector of claim 1, wherein the microparticle comprises a cationic lipid, a polymer of a natural or synthetic monomer, an anonic surfactant or a combination thereof.

11. The viral vector of claim 1, wherein the microparticle further comprises polyvinyl alcohol, polyvinyl pyrilidone, carboxymethyl cellulose, gelatin, polyoxyethylene(20) sorbitan monolaurate or a combination thereof.

12. The viral vector of claim 1, which is conjugated to the microparticle by surface adsorption.

13. The viral vector of claim 1, which is conjugated to the microparticle by covalent interaction.

14. The vital vector of claim 1, which is conjugated to the microparticle by non-covalent interaction.

15. The viral vector of claim 1, which is conjugated to the microparticle by ionic interaction.

16. The viral vector of claim 1, wherein the polypeptide i, an antigen associated with cancer, autoimmune disease, or infectious disease.

17. The viral vector of claim 1, wherein the polypeptide is an antigen associated with M. tuberculosis.

18. A method of producing a microparticle for delivery of a viral vector comprising conjugating a viral vector to a microparticle, wherein the microparticle is formed by dissolving a polymer in a solvent solution, emulsifying the solution, and hardening the microparticles formed by the solution.

19. A microparticle produced by the method of claim 18.

20. A method of stimulating cytokine production or proliferation of T cells specific for an antigen, the method comprising contacting the antigen-specific T cells with a vital vector of claim 1, wherein the heterologous immunogenic polypeptide comprises the antigen to which the T cells are specific.

21. A method for delivering a polynucleotide to a cell comprising contacting the cell with a viral vector of claim 1.

22. The method of claim 21, wherein the cell is an antigen-presenting cell.

23. The method of claim 22, wherein the antigen-presenting cell is a dendritic cell.

24. The method of claim 21, wherein the contacting occurs ex vivo.

25. The method of claim 21, wherein the contacting occurs in vivo.

26. A vaccine comprising the viral vector of claim 1.

27. The vaccine of claim 26, further comprising an adjuvant.

28. A method for delivering a polynucleotide to a subject comprising administering to the subject a vaccine of claim 26.

29. A method of stimulating an immune response in a subject comprising administering a vaccine of claim 26 to the subject.
